(19) United States
(12) Patent Application Publication
(10) Pub. No.: US 2006/0195108 A1
(43) Pub. Date: Aug. 31, 2006

(54) Patellar bone tunneling system

(76) Inventor: Michael D. Fox, Cedar Park, TX (US)

Correspondence Address: STURM & FIX LLP, 206 SIXTH AVENUE, SUITE 1213, DES MOINES, IA, 50309-4076, US

(21) Appl. No.: 11/391,128

(22) Filed: Mar. 28, 2006

Related U.S. Application Data
(63) Continuation-in-part of application No. 11/362,301, filed on Feb. 24, 2006.
(60) Provisional application No. 60/656,574, filed on Feb. 25, 2005.

(51) Int. Cl. A61B 17/16

(52) U.S. Cl. 606/79

(57) ABSTRACT

A drill guide assembly enabling a surgeon to drill a plurality of parallel tunnels in a patella in order to repair a patellar or quadriceps tendon rupture. The drill bit used with the drill guide assembly doubles as a needle for drawing the sutures through the tunnels after the tunnels are drilled. The drill guide assembly clamps securely on the patella while the tunnels are drilled and the sutures pulled. The drill guide holes are made open to one surface of the drill guide so the sutures can be freed from the drill guide after being pulled.

### CROSS-REFERENCE TO RELATED APPLICATIONS

This application is a continuation-in-part of U.S. Ser. No. 11/362,301 filed Feb. 24, 2006, entitled D-TAIL PATELLAR BONE TUNNELING SYSTEM, which contains disclosure from and claims the benefit under Title 35, United States Code, §119(e) of the following U.S. Provisional Application: U.S. Provisional Application Ser. No. 60/656,574 filed Feb. 25, 2005, entitled D-TAIL PATELLAR BONE TUNNELING SYSTEM.

### STATEMENT REGARDING FEDERALLY SPONSORED RESEARCH OR DEVELOPMENT

Not Applicable

### BACKGROUND OF THE INVENTION

1. Field of the Invention

This invention relates generally to a medical device, and more particularly to a method and apparatus to aid in repairing a patellar or quadriceps tendon rupture.

2. Background Art

The U.S. Provisional Application Ser. No. 60/656,574 filed Feb. 25, 2005, entitled D-TAIL PATELLAR BONE TUNNELING SYSTEM is hereby incorporated by reference.

According to the article written by Dr. Bernard C. Ong and Dr. Orrin Sherman entitled Acute Patellar Tendon Rupture: A New Surgical Technique in the Journal of Arthroscopic and Related Surgery, vol. 16 No. 8, patellar tendon rupture is usually remedied by making three longitudinal, parallel tunnels through the patella. Sutures are pulled through the tunnels, and connected to the tendon. The sutures are pulled tight, providing positive tendon to bone contact.

The aforementioned article, Acute Patellar Tendon Rupture: A New Surgical Technique, is hereby incorporated by reference.

Another article describing the above procedure, also hereby incorporated by reference, is Quadriceps Tendon Rupture by Dr. Doron I. Ilan, Dr. Mitchell Keschner, and Dr. Matthew Leibman, published in the Journal of the American Academy of Orthopaedic Surgions, vol. 11 No. 3. The tunnels are described as being two millimeter diameter holes. The sutures are tied with the knee in full extension.

A drill guide assembly was disclosed in U.S. Pat. No. 4,920,958 for use in arthroscopic surgery. This drill guide provides a means for drilling a single hole before repositioning. It must be disassembled and removed before the suture may be threaded into the eyelet of the associated pin.

A drill guide assembly for producing a bone tunnel is disclosed by Chan in U.S. Pat. No. 6,120,511. The design of this assembly makes it difficult to align the distal end of the unit to the target exit point on the uninjured pole of the patella.

Neither of the drill guide devices disclosed in the above two U.S. Patents were designed for aiding the repair of patellar and quadriceps tendons. Additionally, securing these devices to the patella would permit the bony structure to rotate on the axis of the two-point axial fixation arrangement. Because an injured patella is a free floating bone with only one attached tendon, the patella becomes very difficult to manipulate with only two points of the drill guide secured.

There is, therefore, a need for a drill guide made for assisting in the repair of patellar and quadriceps tendons providing solid securing. There is a further need for a drill guide making it possible to drill a plurality of parallel tunnels without removing the drill guide. Additionally, there is a need for a drill guide that may be left attached while the sutures are pulled, with provision to remove the drill guide and free the sutures from the drill guide. There is another need for a drill guide wherein the drill bit is used to pull the sutures through the tunnels.

### BRIEF SUMMARY OF THE INVENTION

The drill guide assembly of the present invention comprises two major components. The first of these components is the drill guide block, comprising a plurality of apertures through which the drill is passed when drilling and pulling sutures. The second component is a retainer which engages a pole of the patella opposite the pole engaged by the drill guide block.

An object of the present invention is to provide a secure drilling jig or guide for drilling a plurality of parallel tunnels for the purpose of repairing a quadriceps or patellar tendon rupture. To effect the securing of the drill guide, a plurality of pins, integral with the drill guide block, engages the injured pole of the patella. At the opposite patellar pole, a plurality of pins associated with the retainer also engages the bony structure. By sliding the retainer relative to the drill guide block, the distance between these sets of pins may be adjusted. Hence, the drill guide assembly may be squeezed down onto the patella for a secure mounting of the drill guide assembly.

The retainer is held in a fixed position relative to the drill guide block by a locking mechanism. The locking mechanism may be released to spread the retainer pins from the drill guide block pins for installation onto or removal from the patella.

Another object of the present invention is to provide a drill guide assembly enabling a practitioner to drill a plurality of parallel tunnels through the patella. In the preferred embodiment, drill guide holes for three parallel tunnels are provided. The drill bit is engaged in one of the drill guide holes in the drill guide block. The drill guide hole comprises a diameter slightly greater than the drill bit, so the drill bit slides easily into the aperture, but is positively guided within the aperture.

An additional object of the present invention is to allow all the plurality of tunnels to be drilled, and the sutures pulled through the tunnels before disengaging the drill guide assembly and removing the same. To effect this object, grooves are provided in the drill guide block that open the drill guide holes along their entire lengths to the region outside the drill guide block. After the sutures are pulled through the apertures and the drilled tunnels, the drill guide assembly is released and removed. The sutures pass through the grooves and are thereby freed from the drill guide block.

Still another object of the present invention is to provide a patellar drill guide system whereby the drill bit is also used to pull the sutures. In the preferred embodiment, the tunnel is drilled and the drill bit pushed through the tunnel while still under power of the drill until a significant length of the drill bit extends out the patella. The drill bit is then removed from the drill and a suture engaged into an eye provided in the back end of the drill bit. The drill bit, with the suture, may now be pulled through the tunnel to pull the suture through the tunnel.

### BRIEF DESCRIPTION OF THE SEVERAL VIEWS OF THE DRAWINGS

FIG. 1 is a side elevation view of the drill guide assembly;

FIG. 2 is a plan view of the drill guide assembly;

FIG. 3 is a rear elevation view of a drill guide block and tower;

FIG. 4 is a top plan view of the drill guide block and tower;

FIG. 5 is a side elevation view of the drill guide block and tower;

FIG. 6 is a top plan view of the retainer;

FIG. 7 is a side elevation view of the retaining guide;

FIG. 8 is the retainer viewed from the receiving end;

FIG. 9 shows the drill bit;

FIG. 10 is a detail of the drill bit;

FIG. 11 is a perspective view of the drill guide block, tower, drill bit, and sutures;

FIG. 12 shows a portion of the skeletal structure of a knee with the drill guide assembly installed for use; and

FIG. 13 shows a portion of the skeletal structure of a knee with the drill guide assembly removed and sutures passed through the tunnels.

### DETAILED DESCRIPTION OF THE INVENTION

With reference now to the various figures in which identical elements are numbered identically throughout, a description of various exemplary aspects of the present invention will now be provided. The preferred embodiment is shown in the drawings and described with the understanding that the present disclosure is to be considered an exemplification of the invention and is not intended to limit the invention to the embodiments disclosed. Any references, herein, to directions will be determined by the associated figure.

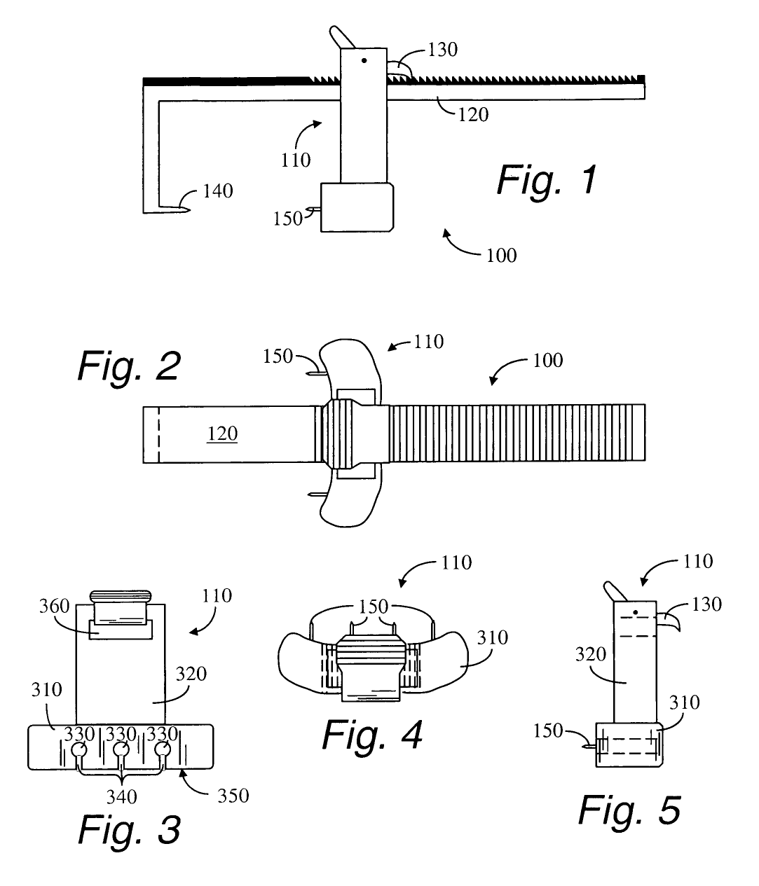

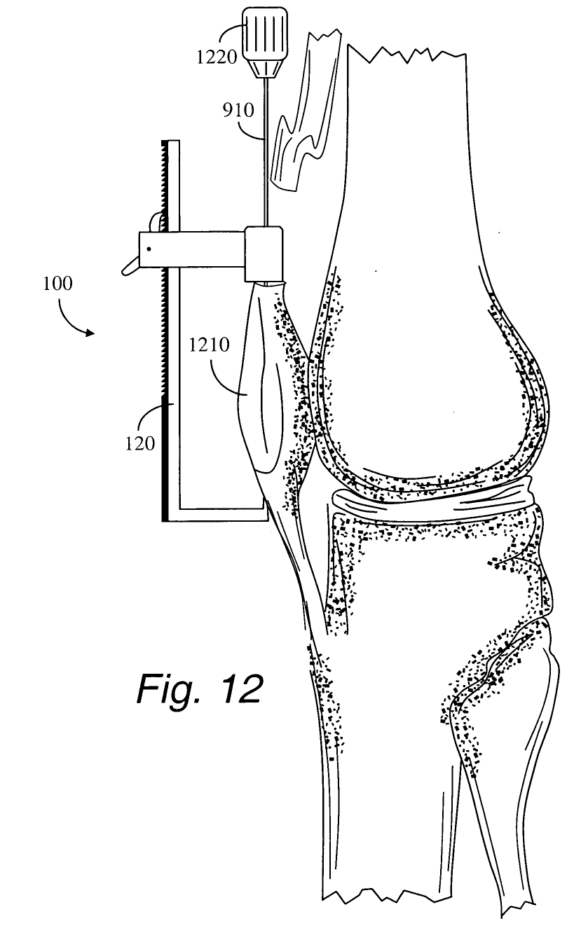

The bone tunneling drill guide assembly 100 is shown assembled in FIGS. 1 and 2 and comprises a drill guide member 110 and a retainer 120. The retainer 120 is slidably engaged to the drill guide member 110 and may be locked in place with a locking mechanism 130. Ultimately, the patella 1210 (see FIG. 12) is engaged between retainer pins 140 and drill guide member pins 150 in use.

The drill guide member 110 is detailed in FIGS. 3-5. The drill guide member 110 comprises a drill guide block 310 and a retainer receiver 320, integral with one another or rigidly connected together. The drill guide block 310 includes three drill guide holes 330 for receiving the drill bit. A groove 340 is formed between each of the three drill guide holes 330 and the bottom surface 350 of the drill guide block 310 to allow for suture 1110 release (see FIG. 11). On the inside of the drill guide block 310, the four drill guide member pins 150 are shown spaced between the guide holes 330. The retainer receiver 320 has an aperture 360 into which the retainer 120 is slidably engaged. At the top of the retainer receiver 320, a spring loaded locking mechanism 130 is provided to lock the retainer 120 at a desired position for use.

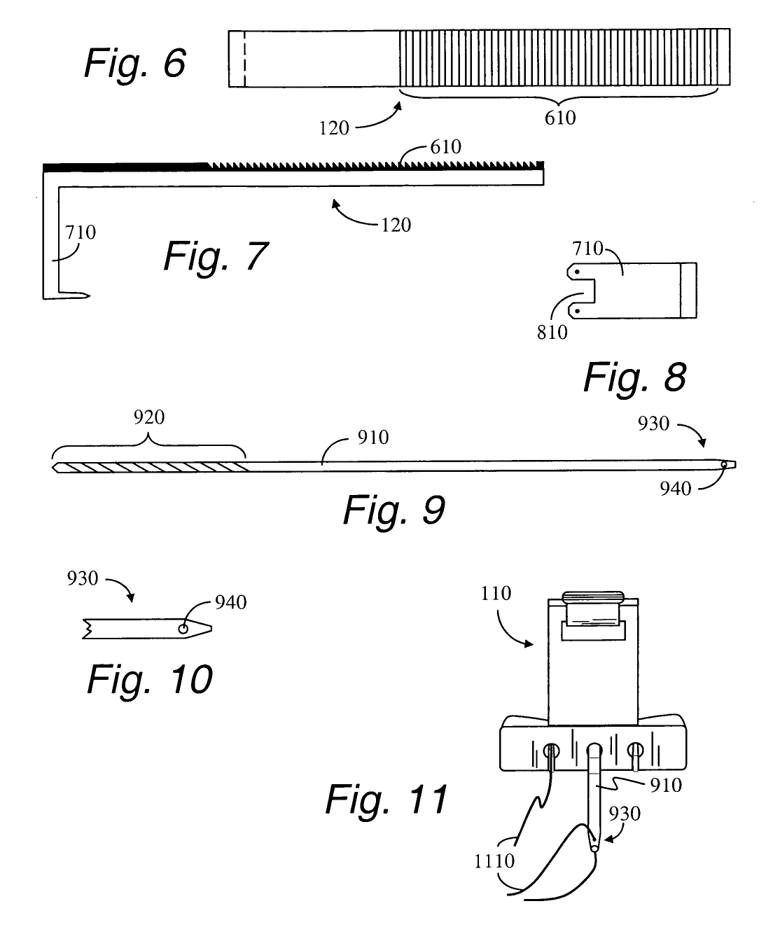

The retainer 120 is detailed in FIGS. 6-8 and comprises locking grooves 610 and an alignment post 710. The plurality of locking grooves 610 encompasses at least a portion of the top of the retainer 120. The locking grooves 610 are engaged by the locking mechanism 130.

The alignment post 710 extends from the receiving end of the retainer 120 downward at a predetermined length. At the lower end of the alignment post 710 there is an exit recess 810 through which the drill bit 910 (see FIG. 9) passes. On the sides of the exit recess 810 are the pointed retainer pins 140.

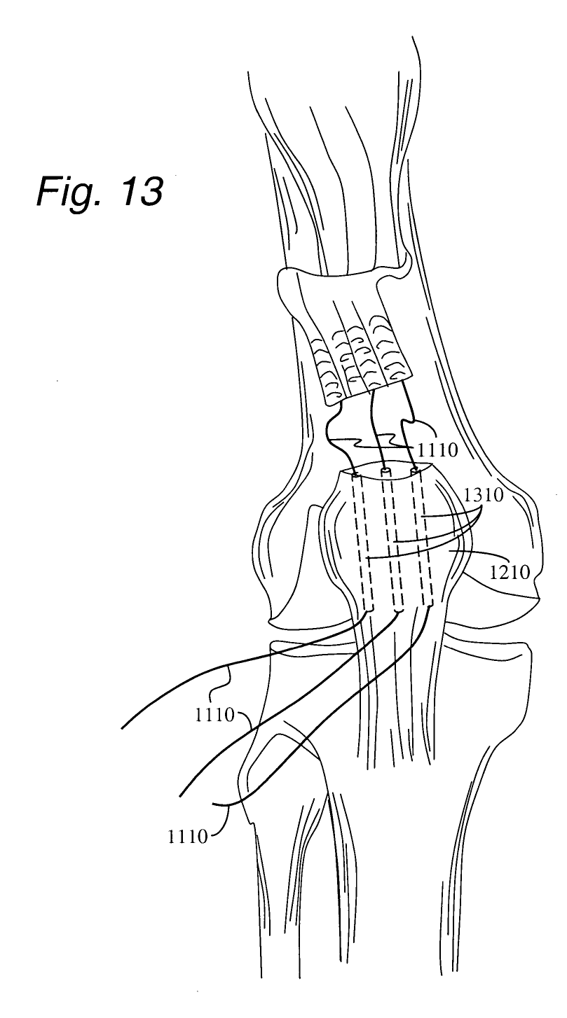

The drill bit 910 comprises three flutes 920 of a predetermined length. The tail 930 is designed with an eye 940 of an appropriate diameter for securing suture 1110, as shown in FIG. 11, in preparation of passing the suture 1110 through the bone tunnels 1310 (FIG. 13). The tail 930 of the drill bit 910 is tapered as detailed in FIG. 10.

In operation the surgeon uses the system of the present invention to create the bone tunnels 1310 necessary and provide a means of suture 1110 passing for the repair of a patellar or quadriceps tendon rupture. Before the surgeon applies this system to the procedure, the approach and soft tissue preparation must first be performed in the usual fashion, well known to those versed in the art. After the approach and soft tissue are prepared, the surgeon assembles the bone tunneling drill guide assembly 100 by placing the retainer 120 and drill guide member 110 together as per FIGS. 1, 2, and 12. The retainer 120 will slide into the aperture 360 of the retainer receiver 320 and begin to lock into the locking mechanism 130. The surgeon will place the retainer pins 140 of the retainer 120 into the uninjured pole of the patella 1210 centered in the anterior-posterior plane as well as in the medial-lateral plane. Then the surgeon will continue locking the bone tunneling drill guide assembly 100 by squeezing the alignment post 710 and the drill guide member 110 toward one another until contact is made between the injured pole of the patella 1210 and the drill guide member pins 150 of the drill guide member 110. The drill guide member pins 150 should also be centered in the anterior-posterior plane as well as in the medial-lateral plane. After proper placement of all the pins 140, 150 has been established, the surgeon must then squeeze the bone tunneling drill guide assembly 100 together a final time to engage all the retaining pins 140, 150 deeper into the patella and complete the locking process. At this point, the bone tunneling drill guide assembly 100 is ready for the insertion of the drill bit 910. The tail end 930 of the drill bit 910 is placed into a pin driver 1220 of a surgical drill and the opposite end of the drill bit 910 is inserted into a receiving end of the drill guide member 110 through one of the three drill guide holes 330. Under the rotation provided by the surgical drill, the drill bit 910 is pushed through the patella 1210 until a generous amount of the fluted end of the drill bit 910 protrudes through the uninjured pole of the patella 1210. The drill bit 910 is then separated from the pin driver 1220 and the suture 1110 is threaded through the eye 940 in the tail 930 of the drill bit 910. The drill bit 910 is now ready for passing the suture 1110 and should be grasped by the fluted end and pulled out of the patella 1210 from the uninjured pole of the patella 1210 using the surgeon's tool of choice such as a pin puller (not shown). This step will pass the suture 1110 through the patellar tunnel 1310, as shown in FIG. 13, releasing it on the proper end for securing. The drill bit 910 is again placed into the pin driver 1220 of the surgical drill and the opposite end of the drill bit 910 inserted into the receiving end of the drill guide member 110 through a second of the three drill guide holes 330 and the process repeated to produce a total of three patellar tunnels 1310. Once three patellar tunnels 1310 have been produced and the sutures 1110 pulled through all three patellar tunnels 1310, the bone tunneling drill guide assembly 100 may be removed from the patella 1210. Upon removal of the bone tunneling drill guide assembly 100, the locking mechanism 130 must first be released to free the retainer 120 from the drill guide member 110. The two components 110, 120 must be separated completely leaving the drill guide member 110 in place on the patella 1210. The sutures 1110 must now be drawn through the grooves 340 at the bottom of the guide holes 330 in order to withdraw the drill guide member 110 from the surgical field. The surgeon may continue the surgical procedure in the usual fashion to complete the repair of a patellar or quadriceps tendon rupture.

I claim:

1. An apparatus used to create bone tunnels for a repair of patellar and quadriceps tendon rupture, the apparatus comprising:
a drill guide member having an upper surface and a lower surface;
a plurality of orifices passing through the drill guide member;
a plurality of grooves, at least one for each orifice, each of said plurality of grooves to provide passage between the respective orifice and the lower surface of the drill guide member;
a retainer receiver operatively, rigidly attached to the upper surface of the drill guide member;
a locking mechanism;
an aperture passing through the retainer receiver in an orientation parallel to the plurality of grooves; and
a retainer operatively, removably, and slidably engaged in the aperture and locked by the locking mechanism to positionally secure the retainer relative to the drill guide member, and comprising an alignment post.

2. The apparatus of claim 1 additionally comprising:
a drill bit having a predetermined length and a first end and a second end;
a plurality of flutes at the first end of the drill bit;
an eye passing diametrically through the second end of the drill bit; and
sutures engagable in the eye of the drill bit.

3. The apparatus of claim 1 additionally comprising a plurality of drill guide member pins, operatively attached to the drill guide member and engagable to a first pole of a patella to provide a secure anchor for the drill guide member.

4. The apparatus of claim 1 additionally comprising a plurality of retainer pins, operatively attached to the retaininer and engagable to a second pole of a patella to provide a secure anchor for the retainer.

5. The apparatus of claim 1 wherein the retainer additionally comprises a plurality of locking grooves, said locking grooves being engaged by the locking mechanism to secure the retainer relative to the drill guide member.

6. The apparatus of claim 3 additionally comprising:
a plurality of retainer pins, operatively attached to the retaininer and engagable to a second pole of a patella to provide a secure anchor for the retainer, said plurality of retainer pins oriented parallel with the plurality of drill guide pins;
a sharp end on each of the plurality of drill guide pins; and
a sharp end on each of the plurality of retainer pins, said sharp ends of the plurality of drill guide pins facing the sharp ends of the plurality of retainer pins.

7. A method of forming parallel bone tunnels with a bone tunneling drill guide assembly, the bone tunneling drill guide assembly comprising a drill guide member having a plurality of drill guide holes therein, a retainer, and a drill bit comprising an eye in a first end of the drill bit, the method comprising:
clamping a patella between the drill guide member and the retainer;
engaging the drill bit into a first of the plurality of drill guide holes;
drilling a first bone tunnel with the drill bit;
engaging a suture in the eye of the drill bit;
pulling the drill bit through the first bone tunnel; and
pulling the suture through the first bone tunnel.

8. The method of claim 7 additionally comprising:
engaging the drill bit into a second of the plurality of drill guide holes;
drilling a second bone tunnel with the drill bit, said second bone tunnel being parallel with the first bone tunnel;
engaging a suture in the eye of the drill bit;
pulling the drill bit through the second bone tunnel; and
pulling the suture through the second bone tunnel.

9. The method of claim 7 wherein clamping the patella between the drill guide member and the retainer comprises:
operatively affixing a plurality of drill guide member pins to the drill guide member;
operatively affixing a plurality of retainer pins to the retainer;
engaging the drill guide member pins to a first pole of the patella;
engaging the retainer pins to a second pole of the patella;
reducing a distance between the drill guide member pins and the retainer pins; and
locking the retainer and the drill guide member relative to one another.

10. The method of claim 7 wherein engaging the drill bit into the first of the plurality of drill guide holes comprises:
engaging the first end of the drill bit in a pin driver; and
engaging a second end of the drill bit, opposite the first end of the drill bit, into the first of the plurality of drill guide holes.

11. The method of claim 7 wherein the drill guide assembly additionally comprises a plurality of grooves, at least one groove associated with each of the plurality of drill guide holes, the grooves opening the drill guide holes to an outside surface of the drill guide member, the method additionally comprising:
removing the drill guide assembly from the patella;
drawing the suture through one of the plurality of grooves to free the suture from the drill guide hole; and
retaining said suture in the first bone tunnel.
